(19) United States
(12) Patent Application Publication
(10) Pub. No.: US 2024/0101520 A1
(43) Pub. Date: Mar. 28, 2024

(54) PYRIMETHAMINE CRYSTAL FORM

(71) Applicant: INTERQUIM S.A. DE C.V., Cuautitlán Izcalli, Estado de México (MX)

(72) Inventors: Abrham Alberto GUTIÉRREZ HERNÁNDEZ, Cuernavaca, Morelos (MX); Aarón RODRÍGUEZ WELTON, Coacalco, Estado de México (MX)

(21) Appl. No.: 18/040,608

(22) PCT Filed: Aug. 6, 2020
(86) PCT No.: PCT/MX2020/000026

§ 371 Date: Feb. 3, 2023

(51) Int. Cl. C07D 239/48

(52) U.S. Cl. CPC C07D 239/48; C07B 2200/13

(57) ABSTRACT

A method for obtaining a new pyrimethamine polymorph, wherein an intermediate a-{4-chlorophenyl)-2-ethyl-1,3-dioxalon-2-acetonitronyl is obtained by means of a reaction with ethylene glycol, and recrystallization is carried out in the presence of dioxane.

### FIELD OF THE DISCLOSURE

The present invention is related to an improved process for the preparation of a new Pyrimethamine polymorph and new intermediates useful in the preparation thereof. In particular, this invention is directed to a new and improved method for preparing Pyrimethamine, which is useful as an antimalarial agent, which can be administered orally, for example, in tablets to humans.

### BACKGROUND

The polymorphs of a drug can present different physicochemical behaviors in their pharmacological properties, for example: density, hardness, water absorption, dissolution rate, thermal stability or behavior in suspension; which can lead to important differences in the efficacy of a pharmaceutical product. Different polymorphs of a substance have the same properties in the liquid or gaseous state, but they behave differently in the solid state. There are two systems of polymorphs nomenclature, the first is based on the melting point of the different isolated and identified polymorphs, thus the roman numeral I identifies the form with the highest melting point, generally the least soluble, the less soluble forms stable in decreasing order of their melting temperature are called II, III, etc.; the second nomenclature is based on the order of discovery, that is, isolation and identification, of the crystalline form using the Greek alphabet, where alpha corresponds to the first discovered form (Sánchez, et. Al., Rev. Mex. of Cien. Farm Vol. 38, 2, (2007) 57-76).

Solvates are called pseudo polymorphism when a solvent is present in the crystalline matrix in stoichiometric proportions, although the terms “solvates” and “hydrates” are more generally used (Pharmacopoeia Arg. Vol. IV. p. 242).

The amorphous phase formation of drugs is desirable to increase their dissolution rate, which results in a significant improvement in their bioavailability (Indian J. Pharm. Sci., 2004, 66(6); 729-734.

Pyrimethamine was discovered in 1952 and was used in medicine in 1953. It is on the World Health Organization's List of Essential Drugs; therefore it is considered a safe and effective drug, necessary in the health system.

Gertrude Elion, an American scientist, a Nobel Prize winner, developed the first drug based on pyrimethamine at Burroughs-Wellcome (now part of GlaxoSmithKline) against malaria, so the drug has been available since 1953. The structure of pyrimethamine is the following:

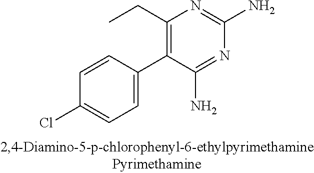 [SMILES: CCc1nc(N)nc(N)c1-c1ccc(Cl)cc1]

Thus, pyrimethamine is a drug used for the treatment of protozoan infections. It is mainly used for the treatment and prophylaxis of malaria and is used in combination with sulfadiazine in the treatment of patients with Toxoplasma gondii infection (toxoplasmosis) in immunocompromised patients, such as in HIV-positive individuals and in the treatment of toxoplasmosis during the 2nd and 3rd trimester of pregnancy. Pyrimethamine interferes with folic acid synthesis by inhibiting the dihydrofolate reductase enzyme (DHFR). Folic acid is required for the synthesis of DNA and RNA in many species, including protozoa.

Pyrimethamine can be prepared in different ways, thus U.S. Pat. No. 2,576,939 (1951) “2,4-DIAMINO-5-PHENYL-6-ALKIL-PYRIMIDINES”, describes a method that initiates from a halo-phenylacetonitrile:

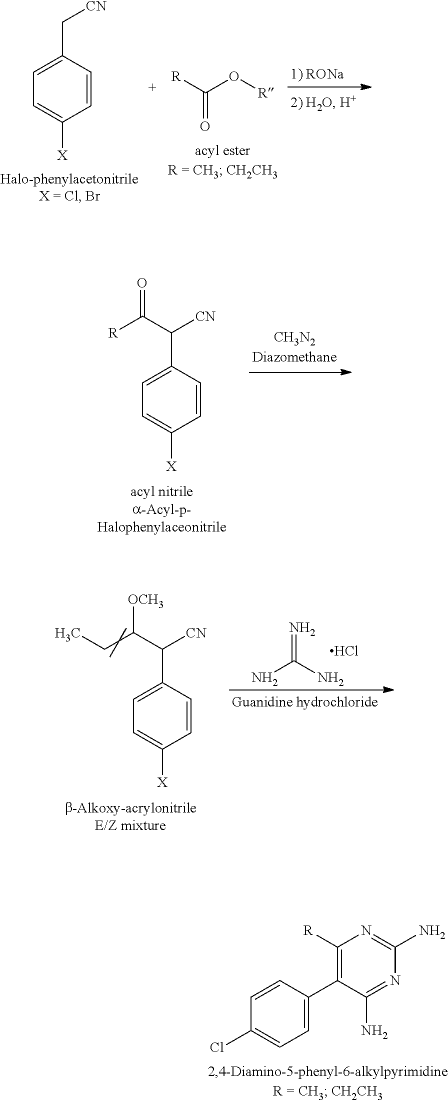

Wherein is not necessary to isolate the acrylonitrile derivative, however, it does not provide acylnitrile synthesis references. The method has the drawback of using Diazomethane as an alkylating agent, since it is an explosive compound and it is not recommended to use it in large quantities.

U.S. Pat. No. 2,602,794 (1952) “PROCESS FOR PREPARATION OF 4-AMINO-5-ARYLPYRIMIDINES”, provides a process consisting of a β-alkoxy-α-aryl-acrylonitrile condensation with a urea derivative, such as guanidine. Various alkylating agents are used in this patent for α-Formylphenylacetonitrile.

| | Yield |
|---|---|
| Alkylation agent | (%) |
| Dimethyl sulfate | 5 |
| Methyl iodide | 11 |
| Ethyl bromide | 13.5 |
| Diazomethane | 60 |
| Ethyl ortho formate | 60 |
| Ethyl ortho n-propionate | 57 |

Example 3 of the patent provides a process for obtaining Pyrimethamine, which follows the sequence:

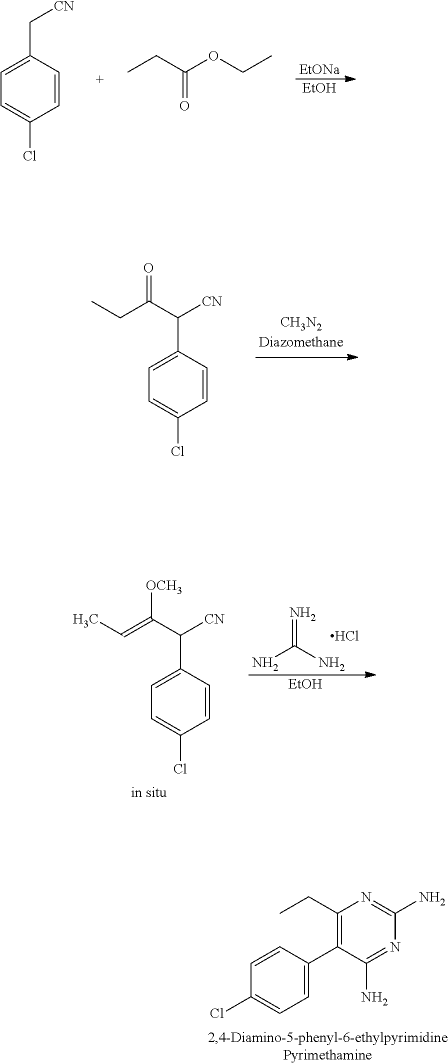

As can be seen in the diagram, the O-alkylation is also carried out with Diazomethane and the acrylonitrile intermediate is not isolated. For the first intermediate (acylnitrile) a melting point of 108-112° C. is reported, which does not agree with the melting point reported in the literature 50-52° C., JACS 73 3763-3770 (1951) and the one obtained in the laboratory by the applicant: 47-50° C. (Fisher). The melting point reported in this patent for Pyrimethamine is 218-220° C.

In U.S. Pat. No. 2,680,740 (1954) “PRODUCTION OF 2,4-DIAMINO-5-(4′-CHLOROPHENYL)-6-ETHYLPYRIMIDINE”, a process that comprises the production of 2,4-Diamino-5-(4′-chlorophenyl)-8-ethylpyrimidine from the condensation of guanidine sulfate with ethyl 2-(4′-chlorophenyl)-3-pentanoate in a reaction medium containing Oleum at a concentration of 15-40% is described, obtaining the 2-Amino-4-hydroxy-5-(4′-chlorophenyl)-6-ethyl pyrimidine, this is treated with phosphorus oxychloride to obtain 2-Amino-4-chloro-5-(4′-chlorophenyl)-6-ethyl pyrimidine, with subsequent treatment with ammonia in ethanol to obtain Pyrimethamine.

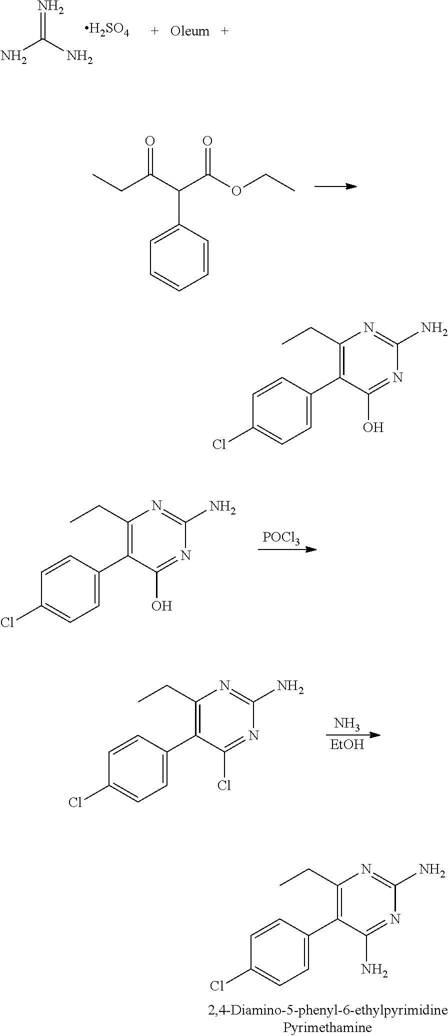

This technique was impractical according to the work of Russell et al. Am. Soc., 73, 3763 (1951).

On the other hand, U.S. Pat. No. 3,939,181 (1976) “INTERMEDIATES FOR THE PREPARATION OF PYRIMIDINE COMPOUNDS”, is directed to a new and improved method for making new intermediates and the preparation thereof. It does not contain reference to the synthesis of α-propionyl-p-chlorophenylacetonitrile. In Example 1, the synthesis of Pyrimethamine is shown, which contains α-propionyl-p-chlorophenylacetonitrle: 131 g; Ethylene glycol: 70 g p-toluenesulfonic acid: 25 g; Toluene: 200 mL. This example does not provide the yield of purified product.

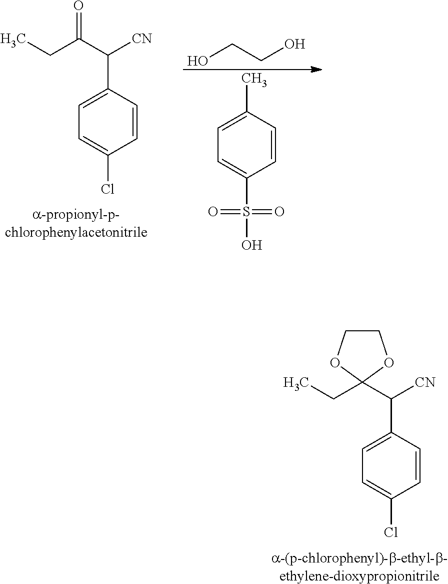

It is known that the solvated and/or crystalline form of any substance with pharmacological activity affect its performance. Furthermore, it is possible that not all polymorphisms and/or solvated forms of the pharmaceutically active ingredient have been described and therefore it is imperative for the pharmaceutical industry to keep up with newly discovered polymorphs and amorphous solvates to avoid problems associated with stability, therapeutic efficacy, safety and preparation.

Different solid forms of pyrimethamine have been described, although new forms have recently been disclosed, whose chemistry in the solid state has been investigated.

A proposal to evaluate the risk associated with the recrystallization of pyrimethamine using ICH II (methanol) and III (acetone, propanol and ethanol) is described in: “The risk of recrystallization: Changes to the toxicity and morphology of Pyrimethamine”. Journal of pharmacy & pharmaceutical sciences, 17, 2, p-190-206, (2014). Anhydrous pyrimethamine saturated solutions were prepared by dissolving in ethanol, methanol, acetone and propanol at solvent reflux temperature. Anhydrous pyrimethamine and methanol solvate pyrimethamine were used in the study, which contains 6% of methanol. The anhydrous pyrimethamine diffractogram, as well as the methanol solvate pyrimethamine diffractogram are shown in FIG. 1.

In this same article, a desolvation test for Pyr-MeOH is described, in which it is observed that the same crystalline form is obtained as anhydrous Pyrimethamine at 473 K (200° C.), FIG. 2.

In “Hydrogen-bonded supramolecular ribbons in the antifolate drug pyrimethamine”. Crystallographic Act Section E, p-817-818, (2002), reference is made to 2 anhydrous forms of Pyrimethamine. It is mentioned that Pyrimethamine is provided by Lupin Laboratories Ltd. India. X-Ray Diffraction is not provided, it only provides the crystalline structure of 2 molecules of pyrimethamine A and B, FIG. 3.

In “A new polymorph and two pseudopolymorphs of pyrimethamine. Crystallographic Act section C, C67 p 428-434, (2011), 2 pseudopolymorphs are disclosed: Pyrimethamine monosolvate of Dimethylsulfoxide (la) and Pyrimethamine monosolvate of N-methylpyrrolidin-2-one (Ib), X-ray diffractograms are not shown, only the crystal structures, FIG. 4.

A study on the crystalline structure of pyrimethamine hydrochloride is found in “Structure of pyrimethamine Hydrochloride”. Analytical sciences, 20, p 175-176, (2004), where only the crystalline structure of pyrimethamine hydrochloride is presented, FIG. 5.

### BRIEF DESCRIPTION OF THE DRAWINGS

FIG. 1. X-ray powder diffractogram. Peaks+2, 2θ; where: A. Anhydrous pyrimethamine, 9.9, 12.9, 18.6, 19.4, 19.9, 20.1, 20.6, 25.3, 28.9 and 29.2; B. Pyrimethamine methanol solvate 8.3, 12.7, 13.9, 14.4, 16.3, 19.0, 20.2, 22.0, 22.6, 23.3 and 28.8.

FIG. 2. Anhydrous pyrimethamine at 473 K (200° C.).

FIG. 3. Crystalline structure of 2 molecules of pyrimethamine A and B.

FIG. 4. Structure of 2 pseudopolymorphs: Dimethylsulfoxide monosolvate pyrimethamine (Ia) and N-methylpyrrolidin-2-one pyrimethamine monosolvate (Ib).

FIG. 5. Crystalline structure of pyrimethamine hydrochloride.

FIG. 6. X-Ray Diffraction of Powders from Lots: DPMET-681808 and DPMET-721809 of Pyrimethamine.

FIG. 7. A. Area from 8 to 12° Theta of the powder X-ray diffractogram of the new polymorph object of the present invention. B. of the Lot: DPMET-681808.

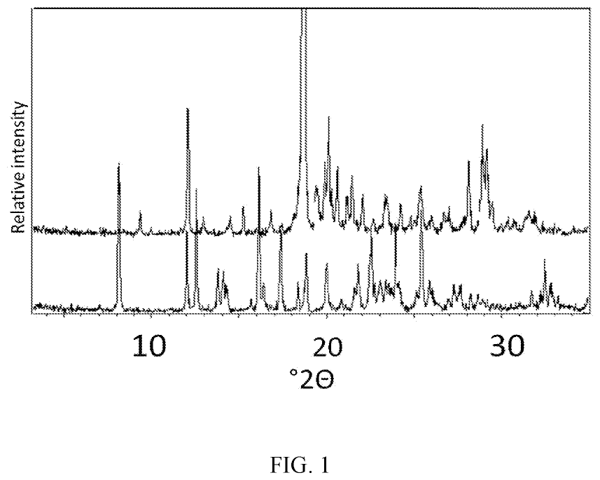

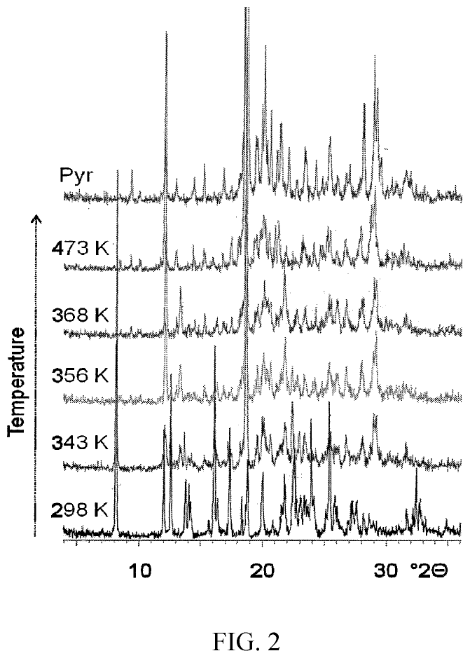

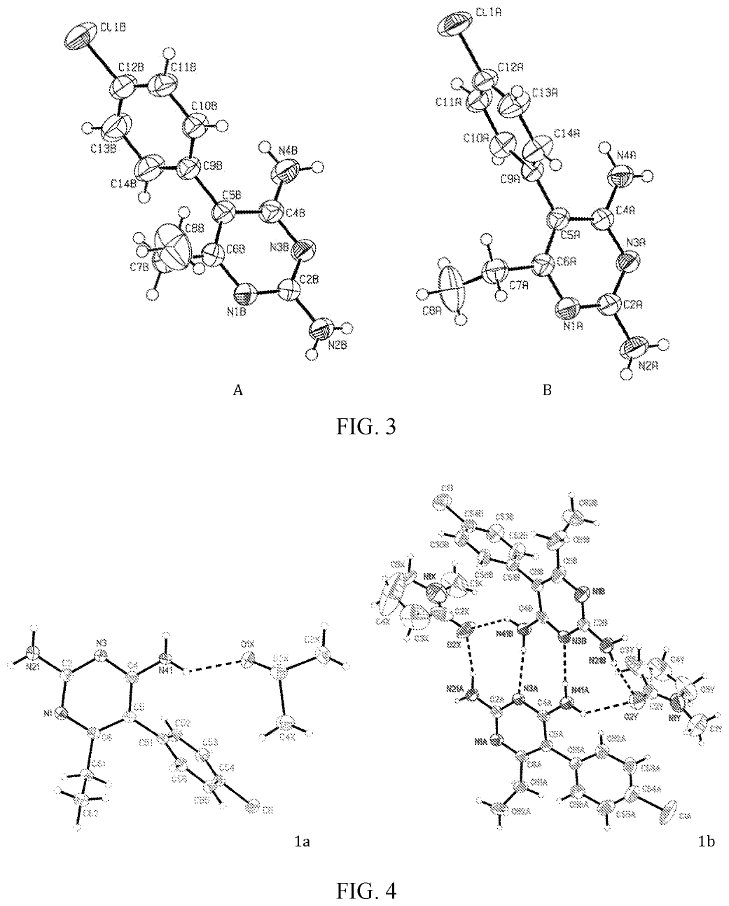

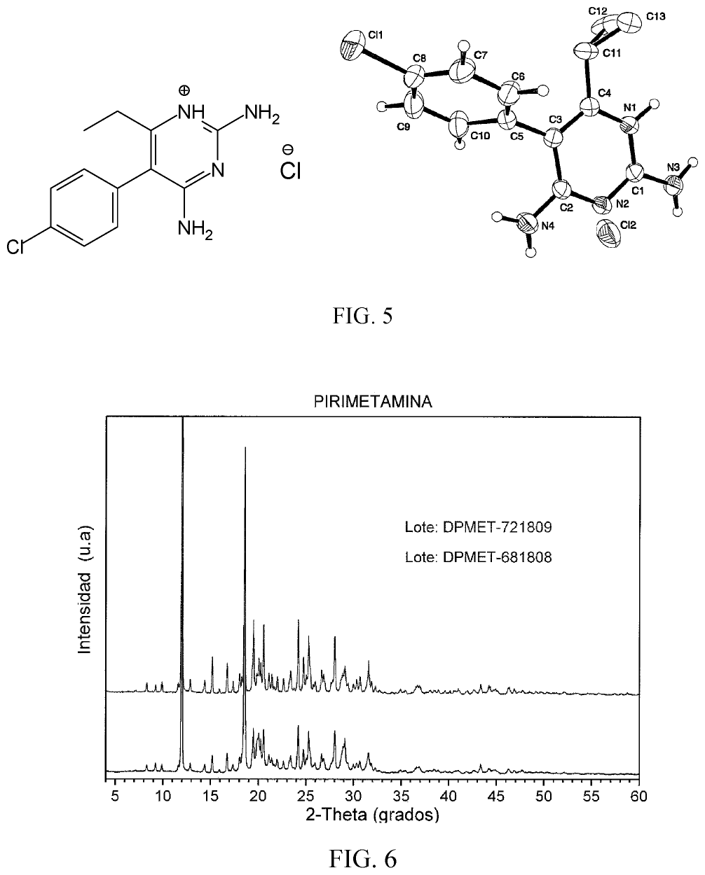

### DETAILED DESCRIPTION

The present invention refers to a new procedure to obtain an amorphous form of Pyrimethamine. The procedure for obtaining amorphous pyrimethamine includes synthesis processes variations disclosed in patents: U.S. Pat. No. 2,602,794 (1952) and U.S. Pat. No. 3,939,181 (1976).

Thus, in example 1 of U.S. Pat. No. 2,602,794 to obtain intermediate 2 in the synthesis of Pyrimethamine, the following changes were introduced.

| | P-toluene | | | | |
|---|---|---|---|---|---|
| | sulfonic | Ethylen | | | Reaction |
| | acid | glycol | Solvent | Yield | time |
| Example | (g) | (g) | mL | (%) | (h) |
| 2 | 8 | (1)* | (1)* | 62 | 2 |
| 3 | 33 | (1)* | (1)* | 97 | 1¼ |
| 4 | (1)* | 83 | (1)* | 98 | 1½ |
| 5 | (1)* | (1)* | Benzene | 93 | 4 |
| | | | 200 | | |
| 6 | (1)* | (1)* | Toluene | 90 | 1 |
| | | | 130 | | |
| 7 | (1)* | (1)* | Toluene | 85 | 1 |
| | | | 300 | | |

(1)* Same conditions of example 1 of U.S. Pat. No. 2,602,794.

In example 14 of U.S. Pat. No. 3,939,181, scaling effect tests are performed.

Example 1 of U.S. Pat. No. 3,939,181, was repeated in different degrees of scaling up.

| Scale (Moles de | Conversion |
|---|---|
| keto nitrile) | (%) |
| 0.6 | 95-100 |
| 5.0 | 95-98 |
| 200.0 | 94-96 |

As can be seen in the table, the yields decrease as the scale increases, but this decrease is not significant.

In Example 15, a comparison of ketal vs enol ether is made.

Consequently, the following changes were made:

1) N-amyl Alcohol (n-Pentanol) is used. In substitution of ethylene glycol.

2) The reaction was carried out at various degrees of scale-up.

| Scale (Moles de | Conversion |
|---|---|
| keto nitrile) | % |
| 0.5 | 95-100 |
| 200.0 | 85 |
| 2000.0 | 70 |

As observed, to higher scale, the performance decreases drastically.

3) Dioxane is used in the recrystallization and then re-pulping in water is carried out. FIG. 6

U.S. Pat. No. 3,939,181 does not refer to the method for obtaining α-propionyl-p-chlorophenylacetonitrile (first intermediate).

Based on the tests carried out, the following synthesis route scheme was established to obtain Pyrimethamine.

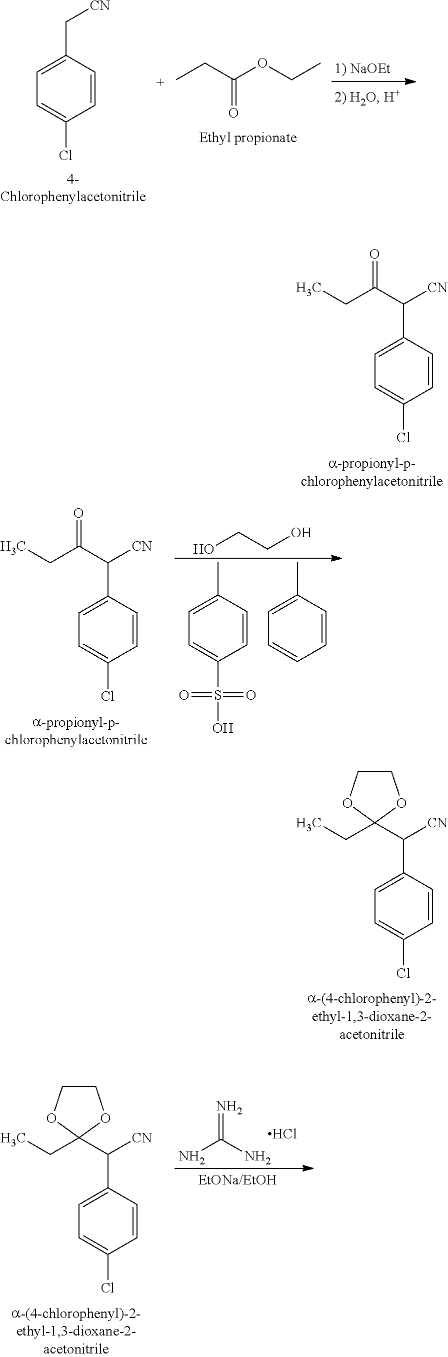

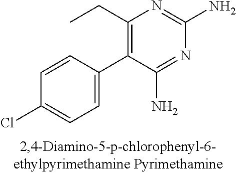 [SMILES: CCc1nc(N)nc(N)c1-c1ccc(Cl)cc1]

### Process of Obtaining Pyrimethamine

### First Intermediate

1.1.—p-Chlorophenylacetonitrile (100 g) is dissolved in anhydrous ethanol (150 mL), once dissolved, 25% sodium methoxide in methanol (150 mL) is added, in an approximate time of 10-15 min.

1.2.—Keep under stirring for 5 minutes and add ethyl propionate (153 mL) and heat at reflux temperature (73±2° C.) for 3.0 h.

1.3.—Cool it at 10±2° C.; the solution is poured into a reactor with Water (700 mL) at 5±2° C. Methylene chloride (300 mL) is added and stirred for 15 min.

1.4.—The organic (lower) and aqueous (upper) phases are separated;

1.5.—Two more extractions are made to the aqueous phase with methylene chloride.

1.6.—The pH of the aqueous phase is adjusted with 20% HCl until pH=7.00±0.05.

1.7.—The neutralized solution is stirred for 10 min.

1.8.—3 extractions are carried out with methylene chloride (300 mL each); the organic phases (lower phase) are combined and dried with anhydrous sodium sulfate.

1.9.—Filter the solution to remove the sodium sulfate.

1.10.—It is distilled to dryness until obtaining a yellow syrup.

### Second Intermediate of Pyrimethamine

2.1.—Toluene (210 mL) is added to the reactor containing the first intermediate and stirred at 25±2° C.

2.2.—Ethylene glycol (35 mL) is added in an approximate time of 10-15 min and left stirring for 5 min.

2.3.—p-toluenesulfonic acid monohydrate (20 g) is added in an approximate time of 10 min., heat to reflux (105±2° C.); produced water is removed using a Dean-Stark trap.

2.4.—Once the reaction is finished, it is cooled to 10±2° C. and washed with water (700 mL) for 15 minutes; the aqueous phase (lower) is discarded and the organic phase (upper) is retained.

2.5.—A wash with 2% NaOH solution (700 mL) is carried out for 15 minutes.

2.6.—Repeat the water wash described in point 2.4, keeping the organic phase.

2.7.—The toluene is distilled until a yellow/amber colored syrup is obtained.

### Obtaining Pyrimethamine

3.1.—Anhydrous ethanol (175 mL) is added to the reactor containing the second intermediate and stirred for 10 minutes at 25±2° C.

3.2.—Simultaneously in another reactor containing anhydrous ethanol (35 mL), guanidine hydrochloride (38 g) is added.

3.3.—Add to the reactor that contains the guanidine hydrochloride, 25% of sodium methoxide in methanol (96 mL) in a time of 5-10 min. and shake for 15 min.

3.4.—Filter the solution over a bed of Celite.

3.5.—The resulting solution (translucent) is added to the reactor of section 3.1 in a time of 5 min.

3.6.—The solution is heated at 56±2° C. until the elimination of methanol; maintain it to reflux for 3.0 h, at a reaction temperature of 73±2° C.

3.7.—Cool it to 10±2° C. and add a mixture of drinking water/Ethanol (350 mL/175 mL); it is kept stirring for 20 minutes.

3.8.—Filter the product and wash it with a 1:1 mixture of water potable:ethanol (70 mL).

3.9.—Let it squeeze for 15 minutes and the product is dried in a vacuum-coupled oven at 70±2° C. for 3 h.

3.10.—45 to 50 grams of crude pyrimethamine are obtained.

### Raw Pyrimethamine Purification

4.—Charge Dioxane (295 mL) and raw Pyrimethamine to a reactor.

4.1.—Heat to 91±2° C. and keep it at this temperature for 10 minutes, until complete dissolution. If necessary, add Dioxane until completely dissolved.

4.2.—Once the dissolution time is over, cool to 10±2° C. and vacuum filter.

4.3.—Wash the pure product with Water (200 mL).

4.4.—Squeeze for 5 minutes and vacuum dry for 1 h at 90° C.+2° C.

4.5.—Re-pulpate the Pyrimethamine in Water (200 mL) at 70±2° C. for 20 min.

4.6.—Cool it at 50±2° C. and filter under vacuum.

4.7.—Wash with a 1:1 solution of Ethanol:Water (100 mL).

4.8.—Squeeze it for 5 minutes and dry in a vacuum oven for 3 h at 80+2° C.

4.9.—40-45 grams of pure pyrimethamine are obtained.

From Powder X-Ray Diffraction (XRD) it is observed that Lots: DPMET-881808 and DPMET-721809 of Pyrimethamine are equivalent.

The obtained pyrimethamine shows a melting point of: 238-240° C. (Fisher), which does not correspond to the melting points reported in the state of the art: 218-220° C. U.S. Pat. No. 2,602,794, 233-234° C., JACS 73 3763-3770 (1951); Merck index, fourteenth edition: 233-234° C. (capillary); 240-242° C. (copper block).

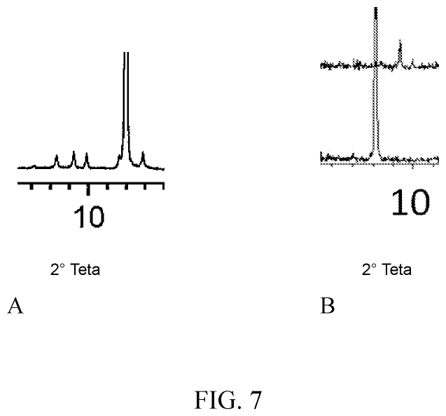

It is demonstrated that the crystallization process provides a new polymorph of Pyrimethamine, this is confirmed mainly in the area of 8 to 12° Theta of the X-Ray Powder Diffraction. FIG. 7.

1. A process for obtaining a new pyrimethamine polymorph comprising the following steps:
a) repairing α-propionyl-p-chlorophenyl acetonitrile from 4-chlorophenylacetonitrile in the presence of sodium methoxide and methanol at a temperature between 73+2° C. for approximately 3 hours;
b) mixing the product of step a) with ethylene glycol in the presence of p-toluenesulfonic acid in toluene to obtain α-(4-chlorophenyl)-2-ethyl-1,3-dioxalon-2-acetonitrile;
c) reacting α-(4-chlorophenyl)-2-ethyl-1,3-dioxalon-2-acetonitrile with guanidine hydrochloride in the presence of hydrochloric acid to obtain Pyrimethamine; and
d) recrystallizing the raw pyrimethamine in dioxane.

2. The process according to claim 1, characterized in that the product from step a) is extracted with methylene chloride water to a pH of 7+0.05, the organic phase is dried with sodium sulfate and it is distilled until a yellow syrup is obtained.

3. The process according to claim 1, characterized in that step b) is carried out at reflux at 105+2° C. until the produced water is eliminated, the reaction mixture is cooled, it is neutralized with NaOH and the organic phase is washed with water, the toluene is distilled until a yellow/amber colored syrup is obtained.

4. The process according to claim 1, characterized in that the product from step c) is dissolved in anhydrous ethanol, it is mixed with a solution of guanidine hydrochloride in anhydrous ethanol and it is stirred for 15 min; it is heated, maintaining reflux for 3.0 h, at a reaction temperature of 73±2° C.; it is cooled at 10±2° C. and is added a mixture of Water/Ethanol and keep stirring; the product is filtered and washed with a 1:1 Water:Ethanol mixture; and it is dried.

5. The process according to claim 1, characterized in that the recrystallization of Pyrimethamine is carried out by heating the mixture with Dioxane at 91±2° C. for 10 minutes; cool it at 10±2° C. and filter under vacuum; wash the product with water and squeeze it for 5 minutes; vacuum dry for 1 h at 90° C.+2° C. and re-pulping in water.

6. The process according to claim 5, characterized in that the re-pulping of Pyrimethamine is carried out in water at 70±2° C. for 20 min; cooling at 50±2° C. and filter under vacuum; wash with a 1:1 solution of Ethanol:Water, squeeze it and dry it in a vacuum oven for approximately 3 hours at 80+2° C.

7. The new polymorph of Pyrimethamine obtained according to the process of claim 1, characterized in that it has a melting point of 238-240° C. (Fisher).

8. The new polymorph according to claim 7, characterized in that it presents the X-ray Powder Diffraction diffractogram shown below:

[symbol]

9. The new polymorph according to claim 7, characterized in that it has values in the area from 8 to 12° Theta of X-ray Powder Diffraction shown below:
